(19) United States
(12) Patent Application Publication
(10) Pub. No.: US 2024/0415011 A1
(43) Pub. Date: Dec. 12, 2024

(54) FUSED RING COMPOUND, SEMICONDUCTOR MATERIAL, AND ELECTRONIC DEVICE

(71) Applicant: Panasonic Intellectual Property Management Co., Ltd., Osaka (JP)

(72) Inventor: Tatsuhito ANDO, Osaka (JP)

(21) Appl. No.: 18/294,685

(22) PCT Filed: Jun. 22, 2022
(86) PCT No.: PCT/JP2022/024968

§ 371 Date: Feb. 2, 2024

(30) Foreign Application Priority Data
Aug. 5, 2021 (JP) 2021-128935

(51) Int. Cl. H10K 85/60; C07C 15/20; C07D 495/04; H10K 10/46

(52) U.S. Cl. CPC H10K 85/625; C07C 15/20; C07D 495/04; H10K 85/6576; C07C 2603/54; H10K 10/46

(57) ABSTRACT

A fused ring compound C of the present disclosure has a fused ring F including a plurality of monocyclic aromatic rings M. The number of the monocyclic aromatic rings M is 11 in the fused ring F. The number of thiophene rings is 2 or less out of the 11 monocyclic aromatic rings M. The fused ring F includes two naphthacene structures. A semiconductor material S according to one aspect of the present disclosure includes the fused ring compound C. An electronic device 10 according to one aspect of the present disclosure includes the semiconductor material S.

### TECHNICAL FIELD

The present disclosure relates to a fused ring compound, a semiconductor material, and an electronic device.

### BACKGROUND ART

In recent years, a lot of electronic devices using a semiconductor film formed from an organic material have been proposed and research and development on them are being conducted actively. Examples of the electronic devices include a thin film transistor (TFT). In the present disclosure, a semiconductor film may be referred to as a semiconductor layer. It is possible to obtain various advantages by using an organic material for the semiconductor layer. For example, conventional inorganic thin film transistors made by using an inorganic material, such as inorganic amorphous silicon, as a base material require a heating process at a temperature of approximately 350° C. to 400° C. when being produced. In contrast, organic TFTs can be produced by a heating process at a low temperature of about 50° C. to 200° C. Therefore, the organic TFTs make it possible to produce a component on a base, such as a plastic film, that has a low heat resistance. In addition, using an organic material has the advantage of making it possible to form a semiconductor layer by using a simple method such as a spin coating method, an ink jet method, or a printing method. According to these methods, a device having a large area can be produced at a low cost.

As one of the indices used to evaluate the performance of a TFT, carrier mobility of a semiconductor layer can be mentioned. A lot of research is being conducted to enhance the carrier mobility of an organic semiconductor layer in an organic TFT. In each of Patent Literatures 1 to 4, for example, research focusing primarily on an organic material for forming an organic semiconductor layer is conducted. Specifically, Patent Literatures 1 to 3 each disclose a condensed cyclic thiophene molecule having a structure in which two thiophene rings and other two to seven monocyclic aromatic rings are condensed. Patent Literature 4 discloses a condensed cyclic thiophene molecule having a structure in which four thiophene rings and other four to nine monocyclic aromatic rings are condensed. An organic semiconductor and organic semiconductor film having good characteristics make it possible to enhance the performance of an electronic device. Therefore, research is required to further enhance the characteristics of organic semiconductors and organic semiconductor films.

Examples of a condensed cyclic thiophene molecule that functions as a p-type organic semiconductor material include benzothieno-benzothiophene (BTBT) and dinaphthothienothiophene (DNTT). These condensed cyclic thiophene molecules are each known as a material that exhibits a relatively high carrier mobility.

### CITATION LIST

### Patent Literature

Patent Literature 1: WO 2017/150474 A1
Patent Literature 2: WO 2016/152889 A1
Patent Literature 3: JP 6318452 B
Patent Literature 4: EP 3050887 A

### SUMMARY OF INVENTION

### Technical Problem

A new fused ring compound suitable for semiconductor materials is required.

### Solution to Problem

A fused ring compound according to one aspect of the present disclosure includes a fused ring including a plurality of monocyclic aromatic rings, wherein
the number of the monocyclic aromatic rings is 11 in the fused ring,
the number of thiophene rings is 2 or less out of the 11 monocyclic aromatic rings, and
the fused ring includes two naphthacene structures.

### Advantageous Effects of Invention

The present disclosure provides a new fused ring compound suitable for semiconductor materials.

### BRIEF DESCRIPTION OF DRAWINGS

FIG. 1 is a schematic structure view showing an example of an electronic device using a fused ring compound of the present disclosure.

FIG. 2 is a schematic structure view showing another example of the electronic device using the fused ring compound of the present disclosure.

### DESCRIPTION OF EMBODIMENTS

(Findings on which the Present Disclosure is Based)

According to the studies of the present inventors, it cannot be considered that conventional condensed cyclic thiophene molecules have a sufficiently high carrier mobility. Therefore, it is difficult to obtain an electronic device with a sufficiently high operating speed by using such conventional condensed cyclic thiophene molecules. In one example, a condensed cyclic thiophene molecule, such as BTBT or DNTT, has a hole mobility of about 5 to 10 cm²/Vs at the highest. In the case where a condensed cyclic thiophene molecule having a hole mobility of this level is used, it is only possible for a component having a gate length of 10 μm to obtain an operating speed of about 1 MHz. Furthermore, even in the case where a component adjusted to have a gate length of about 1 μm is used, an operating speed of about 10 MHz can only be obtained. Note that a gate length of about 1 μm corresponds to the lower limit of a gate length attainable using a coating method. Therefore, in the field of RF-ID (Radio Frequency Identifications) where an operating speed of about 100 MHz is required, an organic semiconductor material having a higher carrier mobility is required.

As a physical quantity that contributes significantly to carrier mobility, reorganization energy is known. The reorganization energy is a physical quantity that depends on the element configuration of a single molecule and the three-dimensional shape of a single molecule. Specifically, the reorganization energy represents an amount of change in energy caused by structural deformation of a molecule when a carrier is conducted by hopping conduction between a plurality of molecules. The smaller the reorganization energy is, the more the carrier mobility of a semiconductor material tends to be enhanced. The semiconductor material with an enhanced carrier mobility makes it possible to achieve an electronic device having a high operating speed.

As a result of intensive studies, the present inventors have newly found that a compound having a specific fused ring tends to have a sufficiently small reorganization energy, and have completed the fused ring compound of the present disclosure.

### (Summary of One Aspect According to the Present Disclosure)

A fused ring compound according to a first aspect of the present disclosure includes a fused ring including a plurality of monocyclic aromatic rings, wherein
the number of the monocyclic aromatic rings is 11 in the fused ring,
the number of thiophene rings is 2 or less out of the 11 monocyclic aromatic rings, and
the fused ring includes two naphthacene structures.

According to the first aspect, the fused ring compound tends to have a sufficiently small reorganization energy and a high carrier mobility. This fused ring compound can be considered suitable for semiconductor materials.

According to a second aspect of the present disclosure, for example, in the fused ring compound according to the first aspect, the 11 monocyclic aromatic rings may be each independently a benzene ring or a thiophene ring.

According to a third aspect of the present disclosure, for example, in the fused ring compound according to the first or second aspect, the fused ring may have a linear structure.

According to a fourth aspect of the present disclosure, for example, the fused ring compound according to any one of the first to third aspects may be represented by formula (I) below:

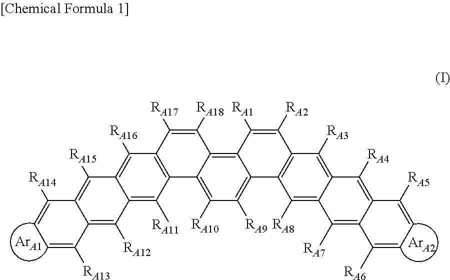

where R_{A1} to R_{A18} are each independently a hydrogen atom or a hydrocarbon group, and Ar_{A1} and Ar_{A2} are each independently a benzene ring that may have a substituent or a thiophene ring that may have a substituent.

According to a fifth aspect of the present disclosure, for example, the fused ring compound according to any one of the first to fourth aspects may be represented by formula (I-1), formula (I-2), formula (I-3), formula (I-4), or formula (I-5) below:

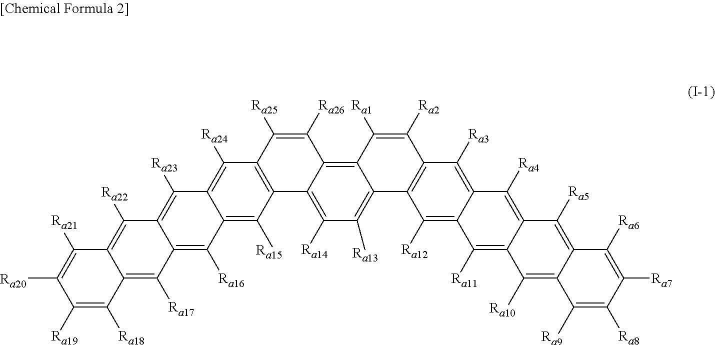

where Rₐ₁ to Rₐ₂₆ are each independently a hydrogen atom or a hydrocarbon group;

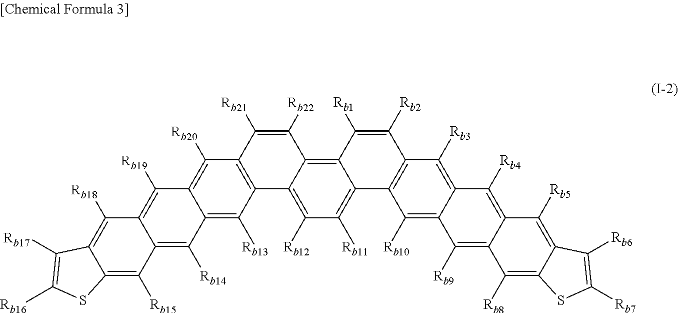

where R_{b1} to R_{b22} are each independently a hydrogen atom or a hydrocarbon group;

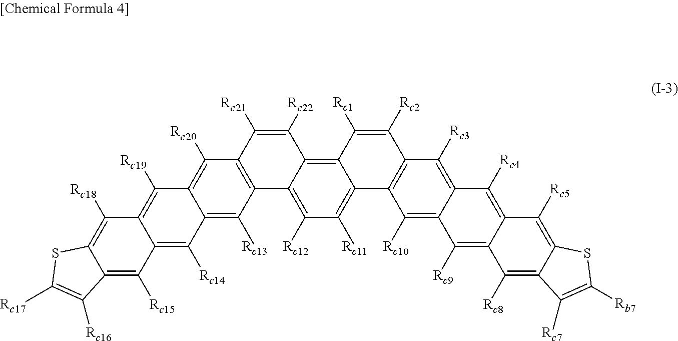

where R_{c1} to R_{c22} are each independently a hydrogen atom or a hydrocarbon group;

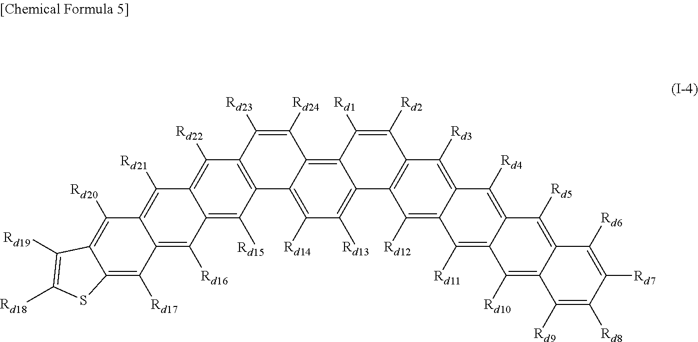

where R_{d1} to R_{d24} are each independently a hydrogen atom or a hydrocarbon group; and

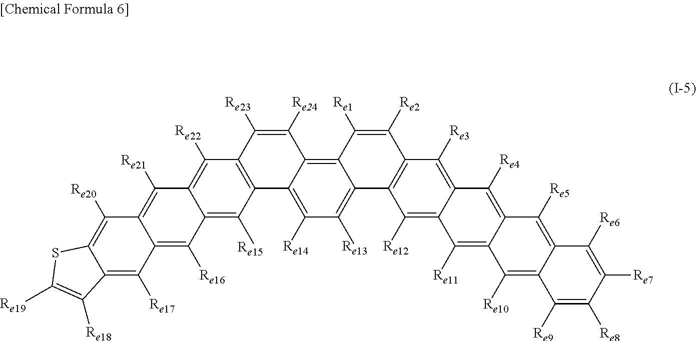

where Rₑ₁ to Rₑ₂₄ are each independently a hydrogen atom or a hydrocarbon group.

The fused ring compounds according to the second to fifth aspects are suitable for semiconductor materials.

According to a sixth aspect of the present disclosure, for example, in the fused ring compound according to any one of the first to fifth aspects, the fused ring may have C_{2V} symmetry.

The fused ring compound according to the sixth aspect tends to be able to be synthesized easily.

A semiconductor material according to a seventh aspect of the present disclosure includes the fused ring compound according to any one of the first to sixth aspects.

According to the seventh aspect, the semiconductor material tends to have a high carrier mobility.

An electronic device according to an eighth aspect of the present disclosure includes the semiconductor material according to the seventh aspect.

According to the eighth aspect, the electronic device tends to have a high operating speed.

An electronic device according to a ninth aspect of the present disclosure includes:
a source electrode;
a drain electrode;
a gate electrode; and
a semiconductor film including the semiconductor material according to the seventh aspect.

According to the ninth aspect, the electronic device tends to have a high operating speed.

Hereinafter, embodiments of the present disclosure will be described with reference to the drawings. The present disclosure is not limited to the following embodiments.

### (Embodiment of Fused Ring Compound)

A fused ring compound C of the present embodiment has a fused ring F including a plurality of monocyclic aromatic rings M. The number of the monocyclic aromatic rings M is 11 in the fused ring F. The number of thiophene rings is 2 or less out of the 11 monocyclic aromatic rings M. The fused ring F includes two naphthacene structures.

Each monocyclic aromatic ring M mean one ring structure having aromaticity. In the present disclosure, the monocyclic aromatic ring M may be simply referred to as “aromatic ring M.” The aromatic ring M typically includes a carbon atom. The aromatic ring M may be composed only of a carbon atom, or may include a hetero atom, such as a sulfur atom, together with a carbon atom. The number of carbon atoms that the aromatic ring M has is not particularly limited and it is 4 or more and 10 or less. As specific examples of the aromatic ring M, a benzene ring and a thiophene ring can be mentioned.

The eleven aromatic rings M may be each independently a benzene ring or a thiophene ring. In one example, the fused ring F may be composed of 11 benzene rings, may be composed of one thiophene ring and ten benzene rings, or may be composed of two thiophene rings and nine benzene rings.

In the present embodiment, when the number of the thiophene rings included in the fused ring F is two or less, the fused ring compound C tends to have a sufficiently high carrier mobility. Furthermore, when the number of the thiophene rings included in the fused ring F is two or less and the number of the aromatic rings M constituting the fused ring F is 11, the fused ring compound C tends to have a sufficiently high carrier mobility. As described later, the fused ring compound C can typically be used as a p-type semiconductor material. Therefore, the carrier mobility of the fused ring compound C may be referred to as hole mobility in the present disclosure.

The eleven aromatic rings M are condensed in the fused ring F. In the present disclosure, the phrase “the aromatic rings M are condensed” means two adjacent aromatic rings M share two carbon atoms and a covalent bond formed between these carbon atoms.

The fused ring F has a linear structure, for example. In the present disclosure, the phrase “the fused ring F has a linear structure” means that the eleven aromatic rings M are arranged in one line without being branched in the fused ring F. That is, each of all the aromatic rings M constituting the fused ring F is condensed with one or two adjacent aromatic rings M. In the fused ring F having a linear structure, there is no aromatic ring M that is condensed with three or more adjacent aromatic rings M. If there is even one aromatic ring M that is condensed with three or more adjacent aromatic rings M, it can be considered that the fused ring F fails to have a linear structure and has a branching structure.

In the fused ring F having a linear structure, the eleven aromatic rings M do not need to be arranged in a straight line. The fused ring F may have a bending structure, for example. In the present disclosure, the phrase “the fused ring F has a bending structure” means that some of the aromatic rings M are arranged in a bent line in the fused ring F.

In the fused ring F having a linear structure, two aromatic rings M present respectively at both ends are each condensed only with one adjacent aromatic ring M. The two aromatic rings M present respectively at both ends may be each independently a benzene ring or a thiophene ring. When the aromatic ring M present at an end is a thiophene ring, the thiophene ring may be condensed with one aromatic ring M that is adjacent thereto in such a manner as to share a 2-position carbon atom and a 3-position carbon atom, or may be condensed with one aromatic ring M that is adjacent thereto in such a manner as to share a 4-position carbon atom and a 5-position carbon atom. Usually, a 1-position atom in a thiophene ring is a sulfur atom.

In the fused ring F having a linear structure, the other aromatic rings M except for the two aromatic rings M present respectively at both ends are each condensed with two adjacent aromatic rings M. When the other aromatic rings M are each a benzene ring, the benzene ring may be condensed with one aromatic ring M that is adjacent thereto in such a manner as to share a 1-position carbon atom and a 2-position carbon atom while being condensed with the other aromatic ring M that is adjacent thereto in such a manner as to share a 3-position carbon atom and a 4-position carbon atom, being condensed with the other aromatic ring M that is adjacent thereto in such a manner as to share a 4-position carbon atom and a 5-position carbon atom, or being condensed with the other aromatic ring M that is adjacent thereto in such a manner as to share a 5-position carbon atom and a 6-position carbon atom.

As described above, the fused ring F includes two naphthacene structures. The naphthacene structures are each represented by formula (1) below. Note that in the two naphthacene structures of the fused ring F, benzene rings constituting the naphthacene structures are not overlapped with each other.

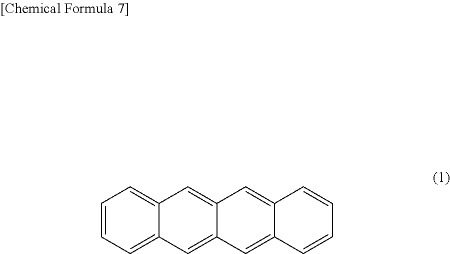 [SMILES: c1ccc2cc3cc4ccccc4cc3cc2c1]

In the present embodiment, because of the fact that the fused ring F has two naphthacene structures, the fused ring compound C tends to have a sufficiently high carrier mobility.

The fused ring F may or may not have symmetry. From the viewpoints that the fused ring compound C can be synthesized easily and production costs can be reduced while the hole mobility of the fused ring compound C can be enhanced further, the fused ring F may have C_{2V} symmetry. The phrase “the fused ring F has C_{2V} symmetry” means that the fused ring F has a two-fold axis of rotational symmetry while having a mirror plane parallel to the axis of rotational symmetry. In the present disclosure, the axis of rotational symmetry may be referred to as a primary axis of the fused ring F. Examples of the fused ring F having C_{2V} symmetry include a structure represented by formula (2) below. In the formula (2), the dashed line indicates the axis of rotational symmetry. At the position of the axis of rotational symmetry, the mirror plane extends in a direction perpendicular to the drawing plane.

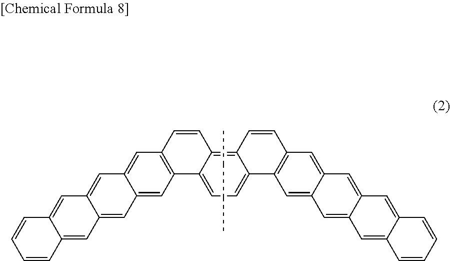

In the fused ring compound C, a hydrogen atom or a substituent is bonded to the fused ring F. The substituent bonded to the fused ring F is not particularly limited. This substituent includes no hetero atom, for example. A specific example of the substituent is a hydrocarbon group. The number of carbon atoms that the hydrocarbon group has is, for example, but not particularly limited to, 1 or more and 20 or less. The hydrocarbon group may be linear, branched, or cyclic. Examples of the hydrocarbon group include an alkyl group and an aryl group.

The number of carbon atoms that the alkyl group has may be 1 or more and 20 or less, 1 or more and 10 or less, or 3 or more and 8 or less. Examples of the alkyl group include a methyl group, an ethyl group (Et), an n-propyl group, an isopropyl group, an n-butyl group, an isobutyl group (i-Bi), a sec-butyl group, a tert-butyl group, a pentyl group, a hexyl group, a heptyl group, an octyl group, a nonyl group, and a decyl group.

The number of carbon atoms that the aryl group has may be 6 or more and 18 or less, or 6 or more and 12 or less. Examples of the aryl group include a phenyl group (Ph), a naphthyl group, a 4-biphenyl group, a 3-biphenyl group, and a 2-biphenyl group.

The fused ring compound C is represented by formula (I) below, for example. The fused ring compound C represented by the formula (I) tends to have a reorganization energy smaller than that of the fused ring compound C represented by the later-described formula (II).

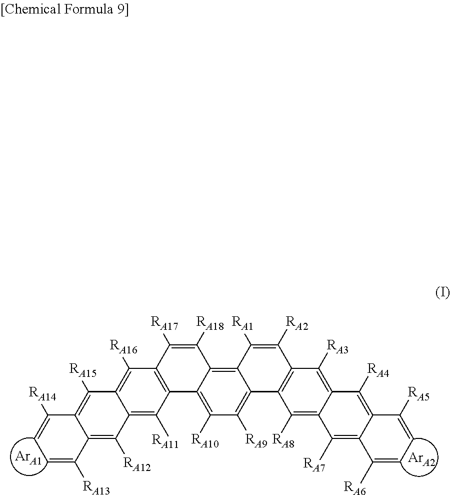

In the formula (I), R_{A1} to R_{A18} are each independently a hydrogen atom or a hydrocarbon group. As the hydrocarbon group, those stated above can be mentioned.

In the formula (I), Ar_{A1} and Ar_{A2} are each independently a benzene ring that may have a substituent or a thiophene ring that may have a substituent. Specific examples of the substituent of the benzene ring and that of the thiophene ring are each a hydrocarbon group. As the hydrocarbon group, those stated above can be mentioned.

The fused ring compound C may also be represented by formula (I-1) below.

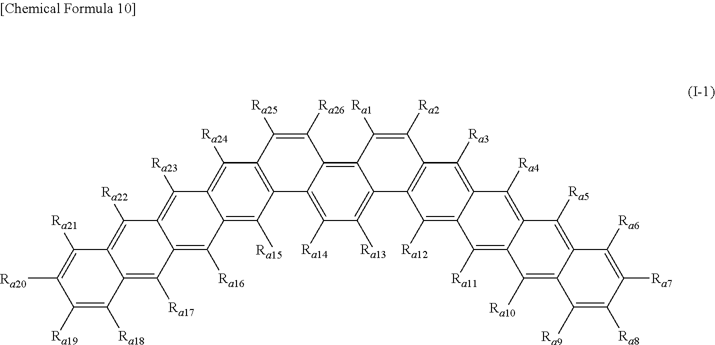

In the formula (I-1), Rₐ₁ to Rₐ₂₆ are each independently a hydrogen atom or a hydrocarbon group. As the hydrocarbon group, those stated above can be mentioned. The fused ring compound C represented by the formula (I-1) is condensed polycyclic hydrocarbon including the fused ring F having a linear structure. In the fused ring compound C, the fused ring F has C_{2V} symmetry.

From the viewpoint of further enhancing the solubility of the fused ring compound C in an organic solvent and the hole mobility of the fused ring compound C, at least one selected from Rₐ₁ to Rₐ₂₆ may be an alkyl group or an aryl group. Rₐ₁ to Rₐ₂₆ may be each independently a hydrogen atom, an alkyl group having 1 to 20 carbon atoms, or an aryl group having 6 to 18 carbon atoms. As the alkyl group and the aryl group, those stated above can be mentioned. Table 1 below shows specific examples of the combination of Rₐ₁ to Rₐ₂₆ in the formula (I-1). In Table 1, the items in the compound column are each an abbreviation of the fused ring compound C having specific Rₐ₁ to Rₐ₂₆.

TABLE 1

| | | Rₐ₂ to | | | | | | Rₐ₁₂ to | | | Rₐ₂₁ to |
|---|---|---|---|---|---|---|---|---|---|---|---|
| Compound | Rₐ₁ | Rₐ₆ | Rₐ₇ | Rₐ₈ | Rₐ₉ | Rₐ₁₀ | Rₐ₁₁ | Rₐ₁₈ | Rₐ₁₉ | Rₐ₂₀ | Rₐ₂₆ |
| i-1 | H | H | H | H | H | H | H | H | H | H | H |
| i-2 | Ph | H | H | H | H | H | H | H | H | H | H |
| i-3 | H | H | Ph | H | H | H | H | H | H | H | H |
| i-4 | H | H | H | 4-biphenyl | H | H | H | H | H | H | H |
| | | | | group | | | | | | | |
| i-5 | H | H | H | H | Et | H | H | H | H | H | H |
| i-6 | H | H | H | H | H | H | Et | H | H | H | H |
| i-7 | H | H | i-Bi | H | H | H | H | H | H | H | H |
| i-8 | H | H | Ph | H | H | H | H | H | H | Ph | H |
| i-9 | H | H | H | Et | H | H | H | H | Et | H | H |

The fused ring compound C may also be represented by formula (I-2) below.

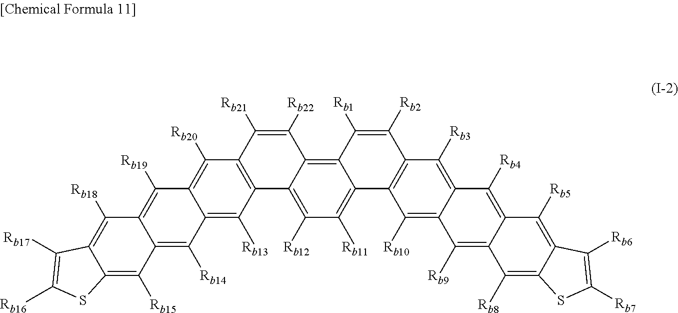

In the formula (I-2), R_{b1} to R_{b22} are each independently a hydrogen atom or a hydrocarbon group. As the hydrocarbon group, those stated above can be mentioned. The fused ring compound C represented by the formula (I-2) is a condensed cyclic thiophene molecule including the fused ring F having a linear structure. In the fused ring compound C, the fused ring F has C_{2V} symmetry.

From the viewpoint of further enhancing the solubility of the fused ring compound C in an organic solvent and the hole mobility of the fused ring compound C, at least one selected from R_{b1} to R_{b22} may be an alkyl group or an aryl group. R_{b1} to R_{b22} may be each independently a hydrogen atom, an alkyl group having 1 to 20 carbon atoms, or an aryl group having 6 to 18 carbon atoms. As the alkyl group and the aryl group, those stated above can be mentioned. Table 2 below shows specific examples of the combination of R_{b1} to R_{b22} in the formula (I-2). In Table 2, the items in the compound column are each an abbreviation of the fused ring compound C having specific R_{b1} to R_{b22}.

TABLE 2

| | R_{b1} to | | | R_{b8} to | | | R_{b18} to |
|---|---|---|---|---|---|---|---|
| Compound | R_{b5} | R_{b6} | R_{b7} | R_{b15} | R_{b16} | R_{b17} | R_{b22} |
| ii-1 | H | H | H | H | H | H | H |
| ii-2 | H | Et | H | H | H | Et | H |
| ii-3 | H | H | Ph | H | Ph | H | H |

The fused ring compound C may be represented by formula (I-3) below.

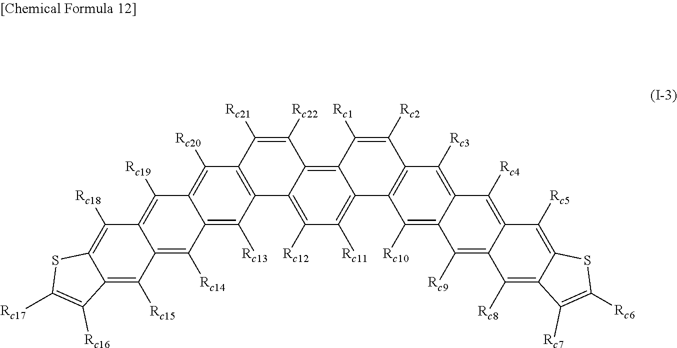

In the formula (I-3), R_{c1} to R_{c22} are each independently a hydrogen atom or a hydrocarbon group. As the hydrocarbon group, those stated above can be mentioned. The fused ring compound C represented by the formula (I-3) is a condensed cyclic thiophene molecule including the fused ring F having a linear structure. In the fused ring compound C, the fused ring F has C_{2V} symmetry.

From the viewpoint of further enhancing the solubility of the fused ring compound C in an organic solvent and the hole mobility of the fused ring compound C, at least one selected from R_{c1} to R_{c22} may be an alkyl group or an aryl group, or may be an alkyl group. R_{c1} to R_{c22} may be each independently a hydrogen atom, an alkyl group having 1 to 20 carbon atoms, or an aryl group having 6 to 18 carbon atoms. As the alkyl group and the aryl group, those stated above can be mentioned. Table 3 below shows specific examples of the combination of Ren to R_{c22} in the formula (I-3). In Table 3, the items in the compound column are each an abbreviation of the fused ring compound C having specific Rei to R_{c22}.

TABLE 3

| | R_{c1} to | | | R_{c8} | | | R_{c18} to |
|---|---|---|---|---|---|---|---|
| Compound | R_{c5} | R_{c6} | R_{c7} | to R_{c15} | R_{c16} | R_{c17} | R_{c22} |
| iii-1 | H | H | H | H | H | H | H |
| iii-2 | H | H | Et | H | Et | H | H |
| iii-3 | H | Ph | H | H | H | Ph | H |

The fused ring compound C may be represented by formula (I-4) below.

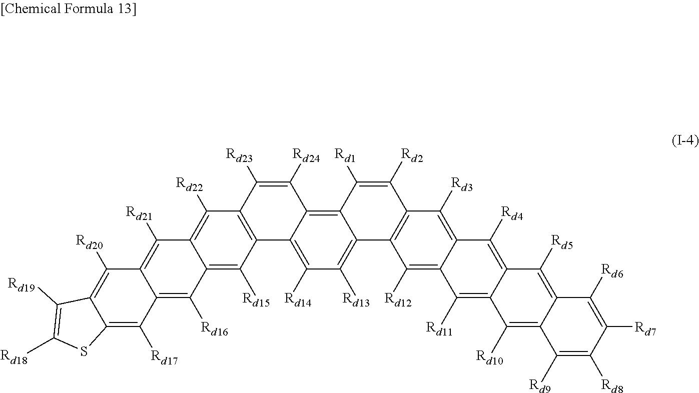

In the formula (I-4), R_{d1} to R_{d24} are each independently a hydrogen atom or a hydrocarbon group. As the hydrocarbon group, those stated above can be mentioned. The fused ring compound C represented by the formula (I-4) is a condensed cyclic thiophene molecule including the fused ring F having a linear structure. In the fused ring compound C, the fused ring F has no symmetry.

From the viewpoint of further enhancing the solubility of the fused ring compound C in an organic solvent and the hole mobility of the fused ring compound C, at least one selected from R_{d1} to R_{d24} may be an alkyl group or an aryl group, or may be an alkyl group. R_{d1} to R_{d24} may be each independently a hydrogen atom, an alkyl group having 1 to 20 carbon atoms, or an aryl group having 6 to 18 carbon atoms. As the alkyl group and the aryl group, those stated above can be mentioned. Table 4 below shows specific examples of the combination of R_{d1} to R_{d24} in the formula (I-4). In Table 4, the item in the compound column is an abbreviation of the fused ring compound C having specific R_{d1} to R_{d24}.

TABLE 4

| Compound | R_{d1} to R_{d24} |
|---|---|
| iv-1 | H |

The fused ring compound C may be represented by formula (I-5) below.

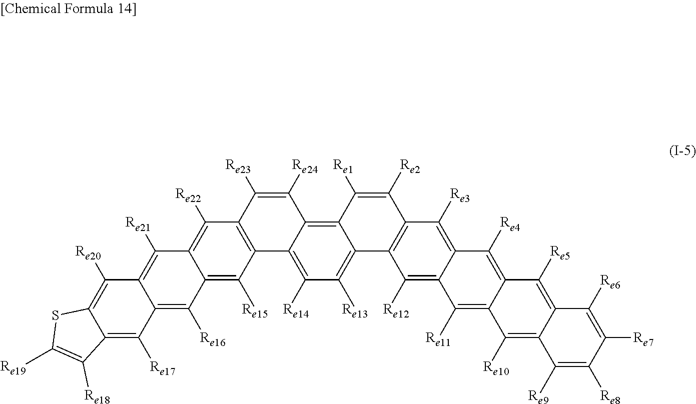

In the formula (I-5), Rₑ₁ to Rₑ₂₄ are each independently a hydrogen atom or a hydrocarbon group. As the hydrocarbon group, those stated above can be mentioned. The fused ring compound C represented by the formula (I-5) is a condensed cyclic thiophene molecule including the fused ring F having a linear structure. In the fused ring compound C, the fused ring F has no symmetry.

From the viewpoint of further enhancing the solubility of the fused ring compound C in an organic solvent and the hole mobility of the fused ring compound C, at least one selected from Rₑ₁ to Rₑ₂₄ may be an alkyl group or an aryl group, or may be Rₑ₁ to Rₑ₂₄ may be each independently a hydrogen atom, an alkyl an alkyl group, group having 1 to 20 carbon atoms, or an aryl group having 6 to 18 carbon atoms. As the alkyl group and the aryl group, those stated above can be mentioned. Table 5 below shows specific examples of the combination of Rₑ₁ to Rₑ₂₄ in the formula (I-5). In Table 5, the item in the compound column is an abbreviation of the fused ring compound C having specific Rₑ₁ to Rₑ₂₄.

TABLE 5

| Compound | Rₑ₁ to Rₑ₂₄ |
|---|---|
| v-1 | H |

The fused ring compound C may be represented by the formula (I-1), the formula (I-2), the formula (I-3), the formula (I-4), or the formula (I-5), or may be represented by the formula (I-1), the formula (I-2), or the formula (I-3). The fused ring compound C represented by the formula (I-1), the formula (I-2), or the formula (I-3) has the fused ring F that is highly symmetric, and tends to be able to be synthesized easily.

The fused ring compound C may be represented by formula (II) below.

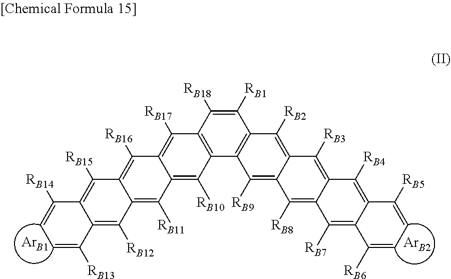

In the formula (II), R_{B1} to R_{B18} are each independently a hydrogen atom or a hydrocarbon group. As the hydrocarbon group, those stated above can be mentioned.

In the formula (II), Ar_{B1} and Ar_{B2} are each independently a benzene ring that may have a substituent or a thiophene ring that may have a substituent. Specific examples of the substituent of the benzene ring and that of the thiophene ring are each a hydrocarbon group. As the hydrocarbon group, those stated above can be mentioned.

The fused ring compound C may be represented by formula (II-1) below.

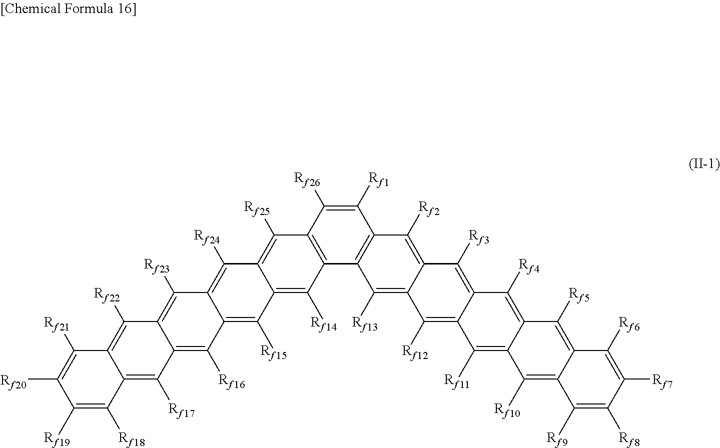

In the formula (II-1), R_{f1} to R_{f26} are each independently a hydrogen atom or a hydrocarbon group. As the hydrocarbon group, those stated above can be mentioned. The fused ring compound C represented by the formula (II-1) is condensed polycyclic hydrocarbon including the fused ring F having a linear structure. In the fused ring compound C, the fused ring F has C_{2V} symmetry.

From the viewpoint of further enhancing the solubility of the fused ring compound C in an organic solvent and the hole mobility of the fused ring compound C, at least one selected from R_{f1} to R_{f26} may be an alkyl group or an aryl group. R_{f1} to R_{f26} may be each independently a hydrogen atom, an alkyl group having 1 to 20 carbon atoms, or an aryl group having 6 to 18 carbon atoms. As the alkyl group and the aryl group, those stated above can be mentioned. Table 6 below shows a specific example of the combination of R_{f1} to R_{f26} in the formula (II-1). In Table 6, the item in the compound column is an abbreviation of the fused ring compound C having specific R_{f1} to R_{f26}.

TABLE 6

| Compound | R_{f1} to R_{f26} |
|---|---|
| vi-1 | H |

The fused ring compound C may be represented by formula (II-2) below.

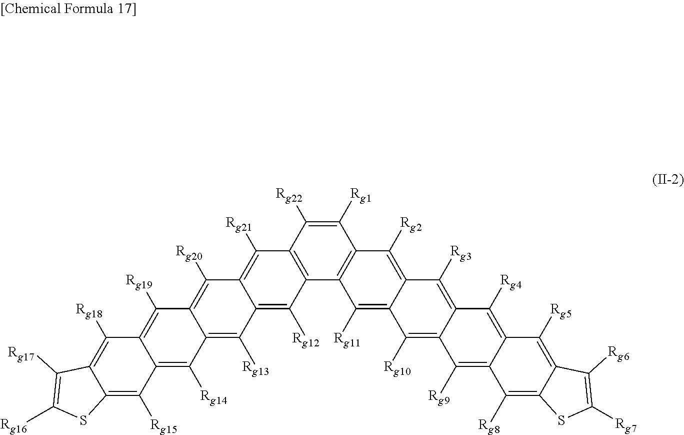

In the formula (II-2), R_{g1} to R_{g22} are each independently a hydrogen atom or a hydrocarbon group. As the hydrocarbon group, those stated above can be mentioned. The fused ring compound C represented by the formula (II-2) is a condensed cyclic thiophene molecule including the fused ring F having a linear structure. In the fused ring compound C, the fused ring F has C_{2V} symmetry.

From the viewpoint of further enhancing the solubility of the fused ring compound C in an organic solvent and the hole mobility of the fused ring compound C, at least one selected from R_{g1} to R_{g22} may be an alkyl group or an aryl group. R_{g1} to R_{g22} may be each independently a hydrogen atom, an alkyl group having 1 to 20 carbon atoms, or an aryl group having 6 to 18 carbon atoms. As the alkyl group and the aryl group, those stated above can be mentioned. Table 7 below shows a specific example of the combination of R_{g1} to R_{g22} in the formula (II-2). In Table 7, the item in the compound column is an abbreviation of the fused ring compound C having specific R_{g1} to R_{g22}.

TABLE 7

| Compound | R_{g1} to R_{g22} |
|---|---|
| vii-1 | H |

The fused ring compound C may be represented by formula (II-3) below.

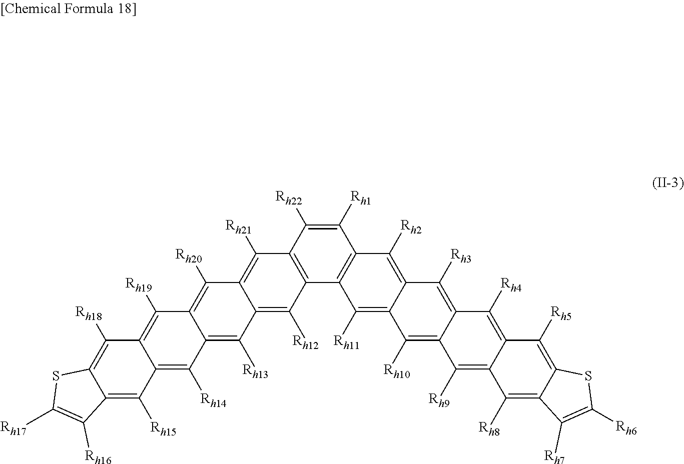

In the formula (II-3), Rₕ₁ to Rₕ₂₂ are each independently a hydrogen atom or a hydrocarbon group. As the hydrocarbon group, those stated above can be mentioned. The fused ring compound C represented by the formula (II-3) is a condensed cyclic thiophene molecule including the fused ring F having a linear structure. In the fused ring compound C, the fused ring F has C_{2V} symmetry.

From the viewpoint of further enhancing the solubility of the fused ring compound C in an organic solvent and the hole mobility of the fused ring compound C, at least one selected from Rₕ₁ to Rₕ₂₂ may be an alkyl group or an aryl group, or may be an alkyl group. Rₕ₁ to Rₕ₂₂ may be each independently a hydrogen atom, an alkyl group having 1 to 20 carbon atoms, or an aryl group having 6 to 18 carbon atoms. As the alkyl group and the aryl group, those stated above can be mentioned. Table 8 below shows a specific example of the combination of Rₕ₁ to Rₕ₂₂ in the formula (II-3). In Table 8, the item in the compound column is an abbreviation of the fused ring compound C having specific Rₕ₁ to Rₕ₂₂.

TABLE 8

| Compound | Rₕ₁ to Rₕ₂₂ |
|---|---|
| viii-1 | H |

The fused ring compound C may be represented by formula (II-4) below.

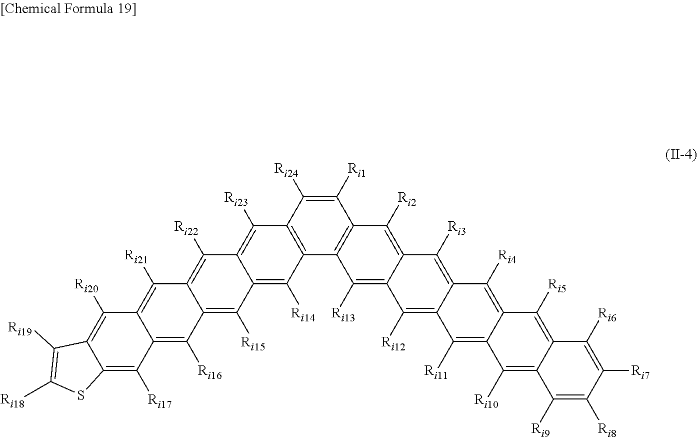

In the formula (II-4), Rᵢ₁ to Rᵢ₂₄ are each independently a hydrogen atom or a hydrocarbon group. As the hydrocarbon group, those stated above can be mentioned. The fused ring compound C represented by the formula (II-4) is a condensed cyclic thiophene molecule including the fused ring F having a linear structure. In the fused ring compound C, the fused ring F has no symmetry.

From the viewpoint of further enhancing the solubility of the fused ring compound C in an organic solvent and the hole mobility of the fused ring compound C, at least one selected from Rᵢ₁ to Rᵢ₂₄ may be an alkyl group or an aryl group, or may be an alkyl group. Rᵢ₁ to Rᵢ₂₄ may be each independently a hydrogen atom, an alkyl group having 1 to 20 carbon atoms, or an aryl group having 6 to 18 carbon atoms. As the alkyl group and the aryl group, those stated above can be mentioned. Table 9 below shows a specific example of the combination of Rᵢ₁ to Rᵢ₂₄ in the formula (II-4). In Table 9, the item in the compound column is an abbreviation of the fused ring compound C having specific Rᵢ₁ to Rᵢ₂₄.

TABLE 9

| Compound | Rᵢ₁ to Rᵢ₂₄ |
|---|---|
| ix-1 | H |

The fused ring compound C may be represented by formula (II-5) below.

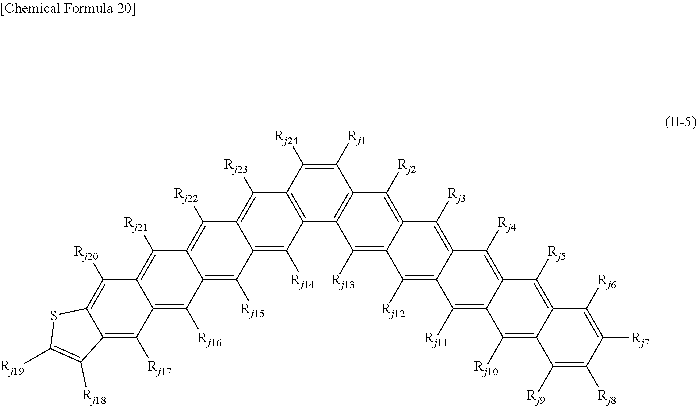

In the formula (II-5), Rⱼ₁ to Rⱼ₂₄ are each independently a hydrogen atom or a hydrocarbon group. As the hydrocarbon group, those stated above can be mentioned. The fused ring compound C represented by the formula (II-5) is a condensed cyclic thiophene molecule including the fused ring F having a linear structure. In the fused ring compound C, the fused ring F has no symmetry.

From the viewpoint of further enhancing the solubility of the fused ring compound C in an organic solvent and the hole mobility of the fused ring compound C, at least one selected from Rⱼ₁ to Rⱼ₂₄ may be an alkyl group or an aryl group, or may be an alkyl group. Rⱼ₁ to Rⱼ₂₄ may be each independently a hydrogen atom, an alkyl group having 1 to 20 carbon atoms, or an aryl group having 6 to 18 carbon atoms. As the alkyl group and the aryl group, those stated above can be mentioned. Table 10 below shows a specific example of the combination of Rⱼ₁ to Rⱼ₂₄ in the formula (II-5). In Table 10, the item in the compound column is an abbreviation of the fused ring compound C having specific Rⱼ₁ to Rⱼ₂₄.

TABLE 10

| Compound | Rⱼ₁ to Rⱼ₂₄ |
|---|---|
| x-1 | H |

### [Method for Producing Fused Ring Compound]

It is possible to synthesize the fused ring compound C by causing a combination of known reactions, such as a halogenation reaction and a Sonogashira coupling reaction, using a commercially available compound as a starting material. When the reactions generate by-products, a separating process and a refining process may be performed by a known method such as column chromatography.

### [Method for Identifying Fused Ring Compound]

The fused ring compound C of the present disclosure can be identified by an elemental analysis, mass spectrometry, and ¹³C-NMR. That is, a ratio of the number of atoms constituting one molecule of the fused ring compound C is specified by an elemental analysis. A molecular weight of the fused ring compound C is specified by mass spectrometry. Based on these results, a molecular formula of the fused ring compound C can be determined. Furthermore, it is possible to determine a structural formula of the fused ring compound C by analyzing the amount of chemical shift at each peak obtained by ¹³C-NMR. These methods also make it possible to specify the type of a substituent and the position of a substituent.

### [Characteristics of Fused Ring Compound]

The fused ring compound C of the present embodiment tends to have a sufficiently small reorganization energy and a high carrier mobility. As for the fused ring compound C, the reorganization energy caused by hole movement is, for example, but not particularly limited to, 0.10 eV or less, and may be 0.08 eV or less, 0.07 eV or less, or 0.06 eV or less. The lower limit of the reorganization energy of the fused ring compound C is, for example, but not particularly limited to, 0.04 eV.

A hopping rate of a charge corresponding to carrier mobility can be calculated by formula (F1) below. The formula (F1) is reported in documents such as David R. Evans et al, Organic Electronics, 2016, Vol. 29, p. 50.

$$[ Equation 1 ]$$

$$K = \frac{π {H}^{2}}{ℏ \sqrt{πλ {k}_{B} T}} exp ( - \frac{λ}{4 {k}_{B} T} ( 1 + \frac{Δ G}{λ} ) ) \qquad ( F1 )$$

In the formula (F1), the symbol κ is a hopping rate of a charge. The symbol ΔG is an amount of change in free energy caused by charge transfer. The symbol λ is a reorganization energy according to Marcus theory. The symbol H is electron coupling between molecules. The symbol k_{B} is a Boltzmann constant. The symbol T is a temperature. The symbol h− (h bar) is a Planck constant.

A reorganization energy is a physical quantity that depends on the element configuration of a single molecule and the three-dimensional shape of a single molecule. Specifically, the reorganization energy represents an amount of change in energy caused by structural deformation of a molecule when a carrier is conducted by hopping conduction between a plurality of molecules. The reorganization energy significantly contributes to the speed of charge transport. The smaller the value of the reorganization energy is, the more the carrier mobility tends to be enhanced. The fact that the reorganization energy and the carrier mobility correlate well with each other is reported in documents such as Shigeyoshi Sakaki et al, J. Phys. Chem. A, 1999, Vol. 103, p. 5551-5556.

The reorganization energy A is defined by formula (F2) below using energy values in four states: E (neutral state in neutral geometry); E* (neutral state in ionic geometry); E_{±} (ionic state in ionic geometry); and E_{±}* (ionic state in neutral geometry).

$$[ Equation 2 ]$$

$$λ = ( {E}_{\pm }^{*} - {E}_{\pm } ) + ( {E}^{*} - E ) \qquad ( F 2 )$$

The reorganization energy λ can be calculated by a density functional method, for example. A known software, such as Gaussian09, can be used for this calculation. Here, B3LYP can be used as a functional. As a basis function, 6-31G (d, p) can be used.

### [Application of Fused Ring Compound]

The fused ring compound C of the present disclosure tends to have an excellent carrier mobility. Therefore, the fused ring compound C is suitable for semiconductor materials. In another aspect, the present disclosure provides a semiconductor material S including the fused ring compound C. The fused ring compound C has an excellent hole mobility, for example. The semiconductor material S including the fused ring compound C as just mentioned can be used as a p-type semiconductor material, for example. In the present disclosure, the semiconductor material S including the fused ring compound C may be referred to as a molecular-based organic semiconductor material or a carbon-based hole transport material.

The semiconductor material S including the fused ring compound C of the present disclosure can be used for electronic devices. In another aspect, the present disclosure provides an electronic device including the semiconductor material S. Specifically, the electronic device includes a semiconductor film including the semiconductor material S. In the present disclosure, the electronic device may be referred to as an electronic component. By using the fused ring compound C for the electronic device, it is possible to enhance the frequency characteristic of the electronic device. As a specific example of the electronic device, a transistor can be mentioned.

### (Embodiment of Electronic Device)

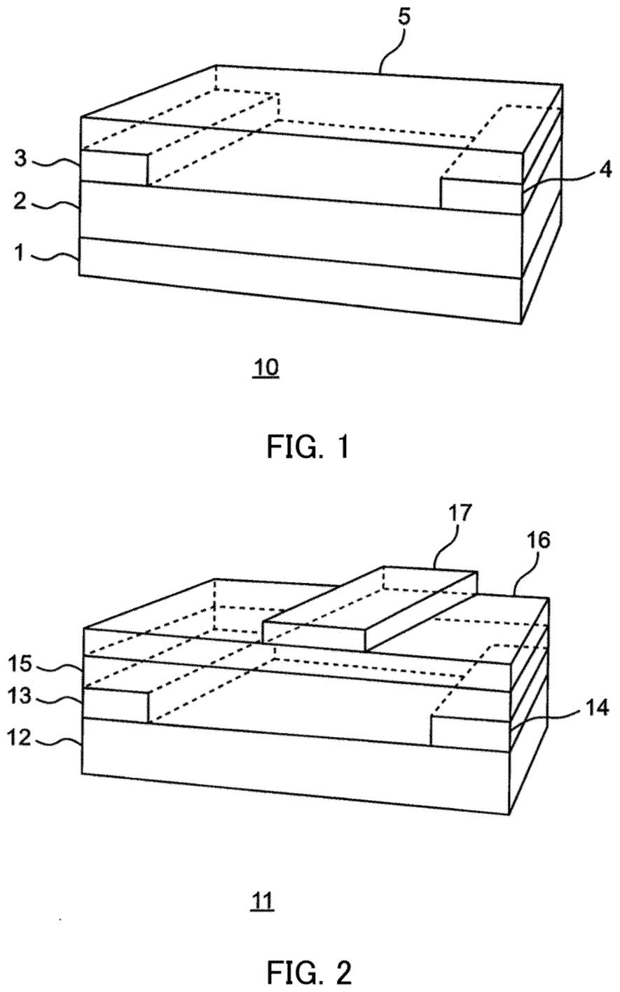

FIG. 1 is a schematic structure view showing an example of an electronic device using the fused ring compound C of the present disclosure. As shown in FIG. 1, an electronic device 10 includes a gate electrode 1, a source electrode 3, a drain electrode 4, and a semiconductor film 5. The electronic device 10 may further include a gate insulating film 2. In the electronic device 10, the semiconductor film 5 includes the semiconductor material S. The electronic device 10 in FIG. 1 is typically a transistor. Specifically, FIG. 1 shows a basic structure of the transistor.

The gate electrode 1 has a plate-like shape, for example, and supports the gate insulating film 2, the source electrode 3, the drain electrode 4, and the semiconductor film 5. The gate insulating film 2 is positioned on the gate electrode 1 and covers a principal surface of the gate electrode 1. The gate insulating film 2 may entirely cover the principal surface of the gate electrode 1.

The source electrode 3 and the drain electrode 4 each have a strip-like shape, for example. The source electrode 3 and the drain electrode 4 are positioned on the gate insulating film 2 in such a manner as not to be in contact with each other. A space is formed between the source electrode 3 and the drain electrode 4. The source electrode 3 and the drain electrode 4 are each in contact with the gate insulating film 2. The source electrode 3 and the drain electrode 4 each extend from one of a pair of end faces of the gate electrode 1 to the other.

The semiconductor film 5 is in contact with each of the gate insulating film 2, the source electrode 3, and the drain electrode 4. Specifically, the semiconductor film 5 covers a surface of the gate insulating film 2 exposed between the source electrode 3 and the drain electrode 4, and also covers each of the source electrode 3 and the drain electrode 4. The semiconductor film 5 fills the space present between the source electrode 3 and the drain electrode 4.

A material of the gate electrode 1 is not particularly limited as long as it is one used as an electrode material in the field of electronic devices. The material of the gate electrode 1 is a metal, for example. Examples of the material of the gate electrode 1 include silicon, gold, copper, nickel, and aluminum.

A material of the gate insulating film 2 is not particularly limited as long as it is one having electrical insulation properties. Examples of the material of the gate insulating film 2 include a metal oxide, a metal nitride, and a polymer material. Examples of the metal oxide include a silicon oxide such as SiO₂, a tantalum oxide such as Ta₂O₅, an aluminum oxide such as Al₂O₃, a titanium oxide such as TiO₂, an yttrium oxide such as Y₂O₃, and a lanthanum oxide such as La₂O₃. Examples of the metal nitride include a silicon nitride such as Si₃N₄. Examples of the polymer material include an epoxy resin, a polyimide (PI) resin, a polyphenylene ether (PPE) resin, a polyphenylene oxide resin (PPO), and a polyvinyl pyrrolidone (PVP) resin.

As materials of the source electrode 3 and the drain electrode 4, the materials stated above for the gate electrode 1 can be mentioned.

As described above, the semiconductor film 5 includes the semiconductor material S. Specifically, the semiconductor film 5 is a p-type semiconductor film including the fused ring compound C. A content of the fused ring compound C in the semiconductor film 5 is, for example, but not particularly limited to, 0.1 mass % or more, and may be 1 mass % or more. From the viewpoint of enhancing the hole mobility, the content of the fused ring compound C may be 10 mass % or more, 50 mass % or more, or 100 mass %.

The semiconductor film 5 may further include an additional material other than the fused ring compound C. Examples of the additional material include fulleren, perylenediimide, polythiophene, and a condensed cyclic thiophene molecule other than the fused ring compound C.

The semiconductor film 5 can be formed by a known method such as a vacuum vapor deposition method or a coating method. When the semiconductor film 5 is formed by a vacuum vapor deposition method, it is possible to form the semiconductor film 5 in which the content of the fused ring compound C is 100 mass % by using only the fused ring compound C as a vapor deposition material.

### (Modification of Electronic Device)

FIG. 2 is a schematic structure view showing another example of the electronic device using the fused ring compound C of the present disclosure. As shown in FIG. 2, an electronic device 11 includes a gate electrode 17, a source electrode 13, a drain electrode 14, and a semiconductor film 15. The electronic device 11 may further include a base substrate 12 and a gate insulating film 16. In the electronic device 11, the semiconductor film 15 includes the semiconductor material S. The electronic device 11 in FIG. 2 is typically a transistor. Specifically, FIG. 2 shows another basic structure of the transistor.

The base substrate 12 has a plate-like shape, for example, and supports the source electrode 13, the drain electrode 14, the semiconductor film 15, the gate insulating film 16, and the gate electrode 17. The base substrate 12 has an insulating layer, for example. The base substrate 12 may have a silicon wafer and the insulating layer that is positioned on the silicon wafer. In the base substrate 12, the insulating layer may entirely cover a principal surface of the silicon wafer.

The source electrode 13 and the drain electrode 14 each have a strip-like shape, for example. The source electrode 13 and the drain electrode 14 are positioned on the base substrate 12 in such a manner as not to be in contact with each other. A space is formed between the source electrode 13 and the drain electrode 14. The source electrode 13 and the drain electrode 14 are each in contact with the base substrate 12, and specifically, they are each in contact with the insulating layer of the base substrate 12. The source electrode 13 and the drain electrode 14 each extend from one of a pair of end faces of the base substrate 12 to the other.

The semiconductor film 15 is in contact with each of the base substrate 12, the source electrode 13, and the drain electrode 14. Specifically, the semiconductor film 15 covers a surface of the base substrate 12 exposed between the source electrode 13 and the drain electrode 14, and also covers each of the source electrode 13 and the drain electrode 14. The semiconductor film 15 is in contact with the insulating layer of the base substrate 12. The semiconductor film 15 fills the space present between the source electrode 13 and the drain electrode 14.

The gate insulating film 16 is positioned on the semiconductor film 15 and covers a principal surface of the semiconductor film 15. The gate insulating film 16 may entirely cover the principal surface of the semiconductor film 15.

The gate electrode 17 has a strip-like shape, for example. The gate electrode 17 is positioned on the gate insulating film 16 and is in contact with the gate insulating film 16. The gate electrode 17 extends from one of the pair of end faces of the base substrate 12 to the other. When the electronic device 11 is viewed in plane, the gate electrode 17 is positioned between the source electrode 13 and the drain electrode 14.

A material of the insulating layer of the base substrate 12 is not particularly limited as long as it is one having electrical insulation properties. Examples of the material of the insulating layer include a metal oxide, a metal nitride, and a polymer material. Examples of the metal oxide include a silicon oxide such as SiO₂, a tantalum oxide such as Ta₂O₅, an aluminum oxide such as Al₂O₃, a titanium oxide such as TiO₂, an yttrium oxide such as Y₂O₃, and a lanthanum oxide such as La₂O₃. Examples of the metal nitride include a silicon nitride such as Si₃N₄. Examples of the polymer material include an epoxy resin, a polyimide (PI) resin, a polyphenylene ether (PPE) resin, a polyphenylene oxide resin (PPO), and a polyvinyl pyrrolidone (PVP) resin.

Materials of the source electrode 13 and the drain electrode 14 are not particularly limited as long as they are ones each used as an electrode material in the field of electronic devices. The materials of the source electrode 13 and the drain electrode 14 are each a metal, for example. Examples of each of the materials of the source electrode 13 and the drain electrode 14 include silicon, gold, copper, nickel, and aluminum.

As described above, the semiconductor film 15 includes the semiconductor material S. Specifically, the semiconductor film 15 is a p-type semiconductor film including the fused ring compound C. A content of the fused ring compound C in the semiconductor film 15 is, for example, but not particularly limited to, 0.1 mass % or more, and may be 1 mass % or more. From the viewpoint of enhancing the hole mobility, the content of the fused ring compound C may be 10 mass % or more, 50 mass % or more, or 100 mass %.

The semiconductor film 15 may further include an additional material other than the fused ring compound C. Examples of the additional material include fulleren, perylenediimide, polythiophene, and a condensed cyclic thiophene molecule other than the fused ring compound C.

A material of the gate insulating film 16 is not particularly limited as long as it is one having electrical insulation properties. As the material of the gate insulating film 16, the materials stated above for the insulating layer of the base substrate 12 can be mentioned.

As a material of the gate electrode 17, the materials stated above for the source electrode 13 and the drain electrode 14 can be mentioned.

### EXAMPLES

Hereinafter, the present disclosure will be described in more detail by Examples. The following Examples describe examples, and the present disclosure is not limited to the following Examples.

### Experimental Example 1

The reorganization energy caused by hole movement was calculated as for dinaphthothienothiophene (DNTT), the compounds (i-1) to (i-9) in Table 1, the compounds (ii-1) to (ii-3) in Table 2, the compounds (iii-1) to (iii-3) in Table 3, the compound (iv-1) in Table 4, the compound (v-1) in Table 5, the compound (vi-1) in Table 6, and the compound (vii-1) in Table 7. DNTT is known as a p-type organic semiconductor material having a high hole mobility. The reorganization energy was calculated by a density functional method based on the formula (F2) mentioned above. Specifically, the reorganization energy was calculated using Gaussian09, which is a calculation software. Here, B3LYP was used as a functional. As a basis function, 6-31G (d, p) was used. Table 11 shows the results.

TABLE 11

| Compound | Reorganization energy [eV] |
|---|---|
| DNTT | 0.12 |
| i-1 | 0.056 |
| i-2 | 0.055 |
| i-3 | 0.057 |
| i-4 | 0.058 |
| i-5 | 0.057 |
| i-6 | 0.058 |
| i-7 | 0.057 |
| i-8 | 0.057 |
| i-9 | 0.057 |
| ii-1 | 0.058 |
| ii-2 | 0.058 |
| ii-3 | 0.062 |
| iii-1 | 0.059 |
| iii-2 | 0.057 |
| iii-3 | 0.058 |
| iv-1 | 0.058 |
| v-1 | 0.058 |
| vi-1 | 0.074 |
| vii-1 | 0.074 |

Table 11 reveals that the fused ring compound C of the present disclosure exhibits a reorganization energy sufficiently lower than that of DNTT. As described above, the reorganization energy and the hole mobility correlate well with each other. Specifically, the reorganization energy and the hole mobility have a negative correlation. From the results of Table 11, it is inferred that the fused ring compound C of the present disclosure has a hole mobility higher than that of DNTT. Accordingly, the fused ring compound C can be considered suitable for semiconductor materials.

### Experimental Example 2

(Production of Compound (i-1))

The compound (i-1) was synthesized in the following manner. First, a tetrachloromethane solution containing commercially available 1-bromo-2,3-dimethylbenzene and bromosuccinimide was prepared. This solution was maintained at a room temperature for 1.5 hours to obtain a compound represented by formula (3) below.

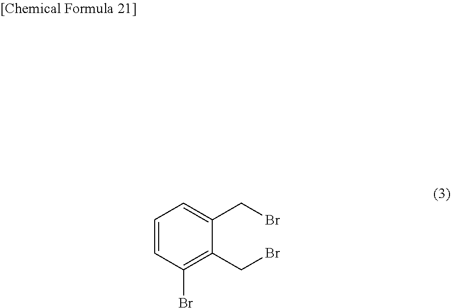 [SMILES: BrCc1cccc(Br)c1CBr]

Next, 1,4-anthraquinone and potassium iodide were added to the obtained compound and the resultant was maintained in N, N-dimethylformamide (DMF) at 110° C. for 20 hours to obtain a compound represented by formula (4) below.

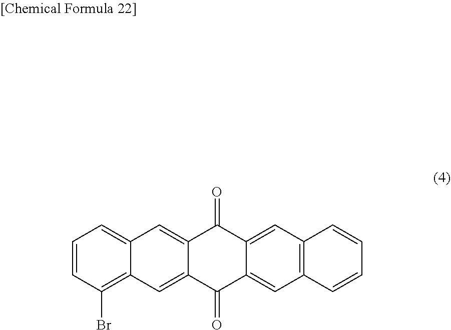 [SMILES: O=C1c2cc3ccccc3cc2C(=O)c2cc3c(Br)cccc3cc21]

Next, aluminum, tetrabromo methane, and mercury (II) chloride were added to the obtained compound and the resultant was maintained in cyclohexanol at 100° C. for about three days. Furthermore, hydrochloric acid and ethanol were added thereto and the resultant was maintained at 100° C. for 2 hours to obtain a compound A represented by formula (5) below.

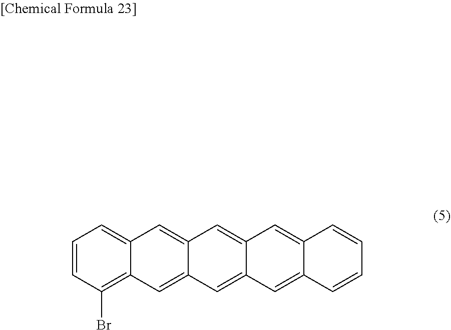 [SMILES: Brc1cccc2cc3cc4cc5ccccc5cc4cc3cc12]

Next, trimethylsilylacetylene, diisopropylamine, CuI, and PdCl₂(PPh₃)₂ were added to the above-mentioned compound A and the resultant was refluxed in tetrahydrofuran for 24 hours. Furthermore, potassium fluoride and methanol were added to the obtained mixed liquid and the resultant was refluxed for 12 hours to obtain a compound represented by formula (6) below.

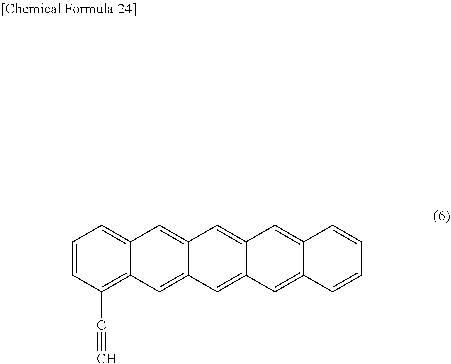 [SMILES: C#Cc1cccc2cc3cc4cc5ccccc5cc4cc3cc12]

Next, the compound A represented by the formula (5), triethylamine, CuI, Pd(PPh₃)₄, and DI-μ-iododicopper were added to the compound represented by the formula (6), and the resultant was maintained in DMF at 55° C. to obtain a compound represented by formula (7) below.

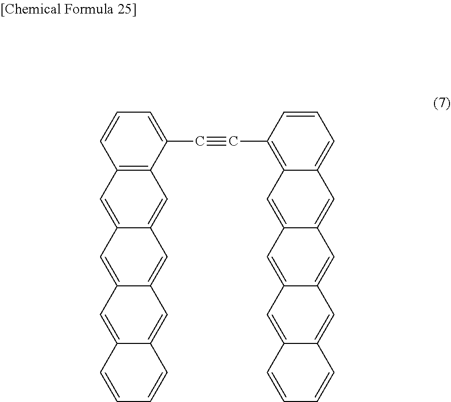 [SMILES: C(#Cc1cccc2cc3cc4cc5ccccc5cc4cc3cc12)c1cccc2cc3cc4cc5ccccc5cc4cc3cc12]

Next, PdCaCo₃, Pd(OCOCH₃)₂, and quinoline were added to the obtained compound, and the resultant was maintained in ethyl acetate under a hydrogen atmosphere at a room temperature for 1 hour to obtain a compound represented by formula (8) below.

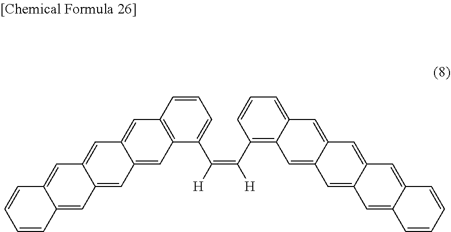 [SMILES: C(=C/c1cccc2cc3cc4cc5ccccc5cc4cc3cc12)/c1cccc2cc3cc4cc5ccccc5cc4cc3cc12]

Next, iodine was added to the obtained compound, and the resultant was maintained in toluene under an oxygen atmosphere at a room temperature for 6 minutes to obtain a compound (i-1) represented by formula (9) below.

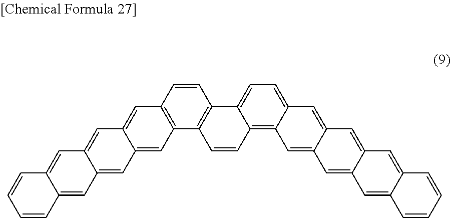 [SMILES: c1ccc2cc3cc4cc5c(ccc6c5ccc5c7cc8cc9cc%10ccccc%10cc9cc8cc7ccc56)cc4cc3cc2c1]

The compound (i-1) was identified by an elemental analysis, mass spectrometry, and ¹³C-NMR. According to the elemental analysis, the ratio between the number of carbon atoms and the number of hydrogen atoms was C:H=23:13. According to the mass spectrometry, the molecular weight was 578.7.

### Experimental Example 3

(Production of Compound (ii-1))

The compound (ii-1) was synthesized in the following manner. First, Ph (OAc)₂ and dichloro(p-cymene)ruthenium(II) were added to commercially available 1-bromoanthraquinone, and the resultant was maintained in trifluoroacetic anhydride at 80° C. for 12 hours to obtain a compound represented by formula (10) below.

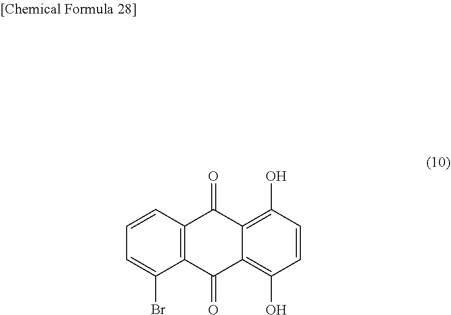 [SMILES: O=C1c2cccc(Br)c2C(=O)c2c(O)ccc(O)c21]

Next, sodium dithionite was added to the obtained compound, and the resultant was maintained in dioxane under a room temperature for about one night to obtain a compound represented by formula (11) below.

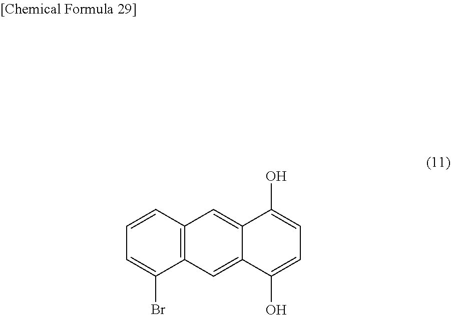 [SMILES: Oc1ccc(O)c2cc3c(Br)cccc3cc12]

Next, 2,3-thiophenedicarboxaldehyde and sodium hydroxide were added to the obtained compound, and the resultant was maintained in a mixed solution of ethanol, water, and tetrahydrofuran at a room temperature for 1 hour to obtain a compound represented by formula (12) below.

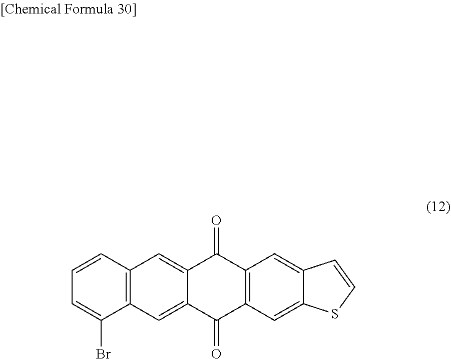 [SMILES: O=C1c2cc3ccsc3cc2C(=O)c2cc3c(Br)cccc3cc21]

Next, sodium borohydride was added to the obtained compound, and the resultant was maintained in tetrahydrofuran at 60° C. for one day or longer. Furthermore, hydrochloric acid and tin (II) chloride were added thereto and the resultant was maintained at 60° C. for 1 hour to obtain a compound B represented by formula (13) below.

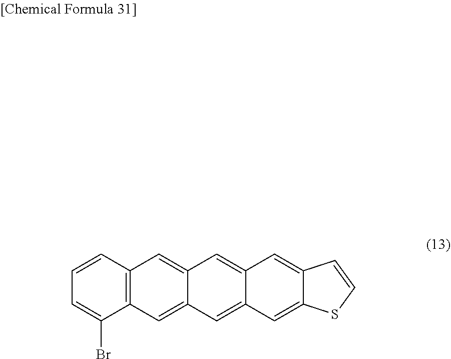 [SMILES: Brc1cccc2cc3cc4cc5ccsc5cc4cc3cc12]

Next, trimethylsilylacetylene, piperidine, CuI, and PdCl2(PPh₃)₂ were added to the above-mentioned compound B, and the resultant was maintained in tetrahydrofuran at 120° C. for 2.5 hours. Furthermore, potassium carbonate, tin chloride, and methanol were added to the obtained mixed liquid, and the resultant was maintained at a room temperature for 40 minutes to obtain a compound represented by formula (14) below.

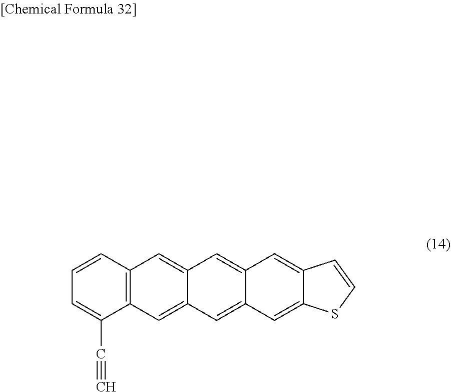 [SMILES: C#Cc1cccc2cc3cc4cc5ccsc5cc4cc3cc12]

Next, the compound B represented by the formula (13), triethylamine, CuI, and PdCl₂(PPh₃)₂ were added to the compound represented by the formula (14), and the resultant was maintained in DMF at 130° C. for 16 hours to obtain a compound represented by formula (15) below.

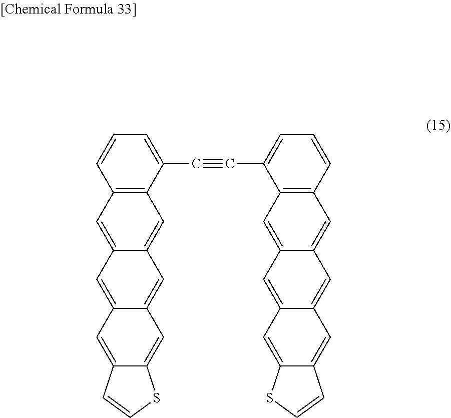 [SMILES: C(#Cc1cccc2cc3cc4cc5ccsc5cc4cc3cc12)c1cccc2cc3cc4cc5ccsc5cc4cc3cc12]

Next, potassium hydroxide and palladium acetate were added to the obtained compound, and the resultant was maintained in DMF at 145° C. for 6 hours to obtain a compound represented by formula (16) below.

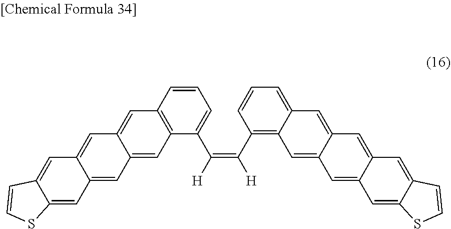 [SMILES: C(=C/c1cccc2cc3cc4cc5ccsc5cc4cc3cc12)/c1cccc2cc3cc4cc5ccsc5cc4cc3cc12]

Next, iron(III) chloride was added to the obtained compound, and the resultant was maintained in a mixed solution of MeNO₂ and CH₂Cl₂ at a room temperature for 1 hour to obtain a compound (ii-1) represented by formula (17) below.

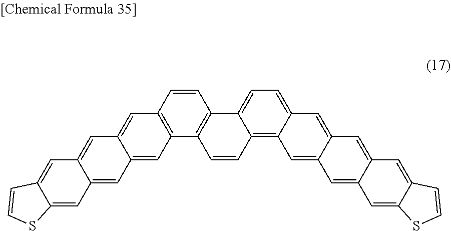 [SMILES: c1cc2cc3cc4cc5ccc6c(ccc7c8cc9cc%10cc%11sccc%11cc%10cc9cc8ccc76)c5cc4cc3cc2s1]

The compound (ii-1) was identified by an elemental analysis, mass spectrometry, and ¹³C-NMR. According to the elemental analysis, the ratio of the number of carbon atoms, the number of hydrogen atoms, and the number of sulfur atoms was C:H:S=21:11:1. According to the mass spectrometry, the molecular weight was 590.756.

### INDUSTRIAL APPLICABILITY

The fused ring compound C of the present disclosure tends to have an excellent carrier mobility. Therefore, the fused ring compound C is suitable for semiconductor materials. Particularly, the fused ring compound C is useful as a p-type semiconductor material. The semiconductor material S including the fused ring compound C can be used for electronic devices. By using the fused ring compound C for an electronic device, it is possible to enhance the frequency characteristic of the electronic device. As a specific example of the electronic device, a transistor can be mentioned.

1. A fused ring compound comprising a fused ring including a plurality of monocyclic aromatic rings, wherein
the number of the monocyclic aromatic rings is 11 in the fused ring,
the number of thiophene rings is 2 or less out of the 11 monocyclic aromatic rings, and
the fused ring includes two naphthacene structures.

2. The fused ring compound according to claim 1, wherein the 11 monocyclic aromatic rings are each independently a benzene ring or a thiophene ring.

3. The fused ring compound according to claim 1, wherein the fused ring has a linear structure.

4. The fused ring compound according to claim 1, wherein the fused ring compound is represented by formula (I) below: 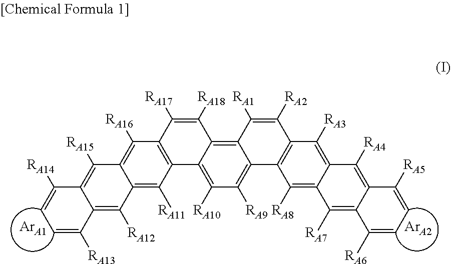
where R_{A1} to RA₁₈ are each independently a hydrogen atom or a hydrocarbon group, and Ar_{A1} and Ar_{A2} are each independently a benzene ring that may have a substituent or a thiophene ring that may have a substituent.

5. The fused ring compound according to claim 1, wherein the fused ring compound is represented by formula (I-1), formula (I-2), formula (I-3), formula (I-4), or formula (I-5) below: 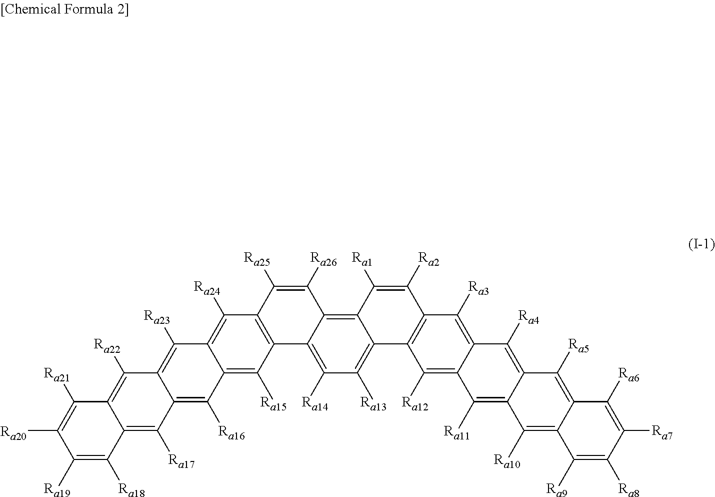
where Rₐ₁ to Rₐ₂₆ are each independently a hydrogen atom or a hydrocarbon group; 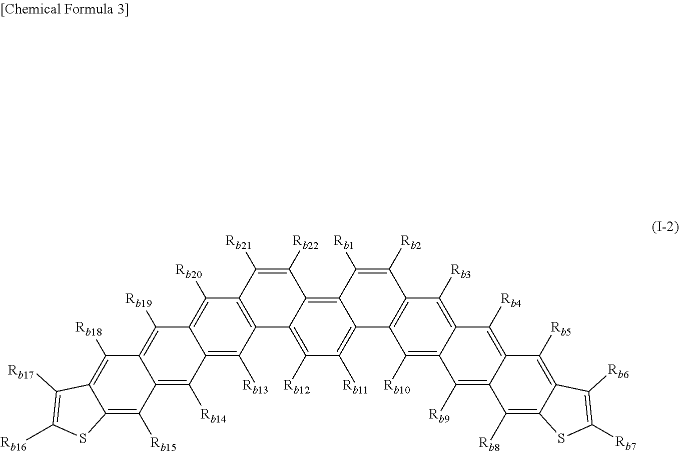
where R_{b1} to R_{b22} are each independently a hydrogen atom or a hydrocarbon group; 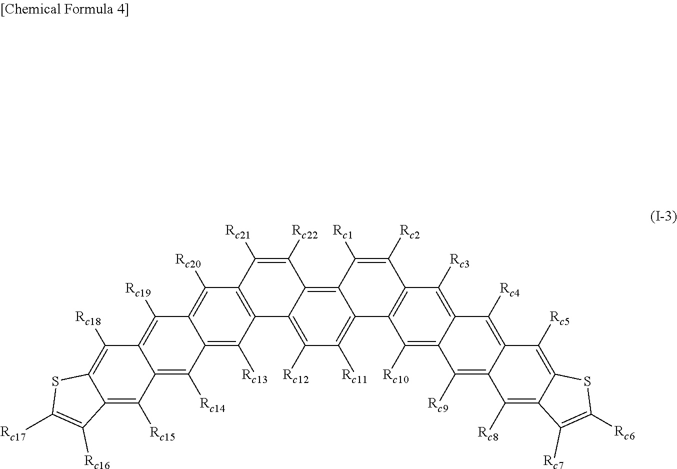
where R_{c1} to R_{c22} are each independently a hydrogen atom or a hydrocarbon group; 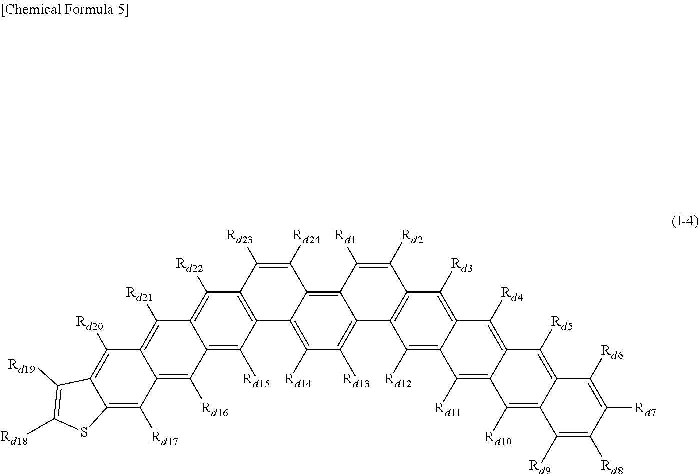
where R_{d1} to R_{d24} are each independently a hydrogen atom or a hydrocarbon group; and 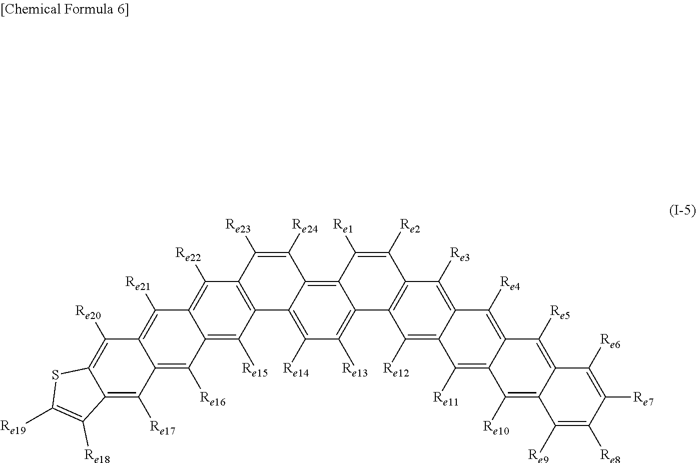
where Rₑ₁ to Rₑ₂₄ are each independently a hydrogen atom or a hydrocarbon group.

6. The fused ring compound according to claim 1, wherein the fused ring has C_{2V} symmetry.

7. A semiconductor material comprising the fused ring compound according to claim 1.

8. An electronic device comprising the semiconductor material according to claim 7.

9. An electronic device comprising:
a source electrode;
a drain electrode;
a gate electrode; and
a semiconductor film including the semiconductor material according to claim 7.
